(19) United States
(12) Patent Application Publication
(10) Pub. No.: US 2026/0176669 A1
(43) Pub. Date: Jun. 25, 2026

(54) PERSONALIZED SKIN ANALYSIS AND SKIN CARE

(71) Applicant: Conopco, Inc., d/b/a UNILEVER, Hoboken, NJ (US)

(72) Inventors: Suzanne Elizabeth ADAMS, Wageningen (NL); Chung-Ching CHU, Wageningen (NL); Xiao CUI, Wageningen (NL); Barry Gerard MURPHY, Wageningen (NL)

(21) Appl. No.: 19/124,815

(22) PCT Filed: Nov. 3, 2023
(86) PCT No.: PCT/EP2023/080720

§ 371 Date: Apr. 25, 2025

(30) Foreign Application Priority Data
Dec. 15, 2022 (WO) PCT/CN2022/139198
Jan. 27, 2023 (EP) 23153591.5

(51) Int. Cl. A61Q 19/00; A61Q 19/08; C12Q 1/6806; C12Q 1/686; C12Q 1/689

(52) U.S. Cl. CPC C12Q 1/6806; A61Q 19/00; A61Q 19/08; C12Q 1/686; C12Q 1/689

(57) ABSTRACT

The invention provides a method for providing skin analysis, and personalized skincare information based on that analysis, to a consumer at a retail point of sale, the method comprising: (i) at the retail point of sale, receiving a sample of the skin microbiome, which is collected from a skin surface of the consumer in a non-invasive manner, (ii) extracting the DNA from the sample of the skin microbiome and analysing the extracted DNA using a portable qPCR thermocycler which is configured to amplify and detect nucleic acids associated with a panel of microorganisms in the sample, (iii) uploading the analysis data from the thermocycler to a machine learning platform, and (iv) providing, at the retail point of sale, a profile of the consumer's skin microbiome and a personalized skincare routine for the consumer, generated from the analysis data using the machine learning platform.

### FIELD OF THE INVENTION

The present invention relates to methods for providing skin analysis, and personalized skin care information based on that analysis, to consumers.

### BACKGROUND OF THE INVENTION

An influx of information online and on social media has educated consumers on their beauty needs and fueled a growing demand for personalized skincare solutions which address their unique concerns.

Efforts have been made to create personalized skincare solutions for consumers, for example by measuring visible skin parameters (such as shine, spots, wrinkles, roughness, and irregular pigmentation) and using the data so acquired to inform product recommendation or design.

Despite the unquestionable value of visual assessment, the phenotypic characteristics being measured are typically end products of the underlying mechanisms which are responsible for healthy skin and its appearance. In order to effectively create personalization in skincare, it is necessary to gain an insight into these underlying mechanisms. This requires an understanding of the skin microbiome and how to maintain its delicate balance. Imbalances in the skin microbiome (dysbiosis) are associated with several skin conditions, either pathological (such as atopic dermatitis, acne vulgaris, psoriasis or lichen planus) or non-pathological (such as sensitive skin, oily skin, or dry skin).

Skin microbiome test kits have been developed for consumer use which generally involve using a sterile swab to collect a sample of the skin microbiome from the skin surface by rubbing the swab over the skin a few times. Then the sample is sent to a lab for DNA extraction and sequencing analysis to determine which microbes are present. Consequently, there may be a considerable time lag, often several days to weeks, between sample collection and analysis.

An objective of the present invention is to provide skin analysis, and personalized skincare information based on that analysis, to consumers at a retail point of sale.

### SUMMARY OF THE INVENTION

The invention provides a method for providing skin analysis, and personalized skin care information based on that analysis, to a consumer at a retail point of sale, the method comprising:
(i) at the retail point of sale, receiving a sample of the skin microbiome, which is collected from a skin surface of the consumer in a non-invasive manner,
(ii) extracting the DNA from the sample and analysing the extracted DNA using a portable qPCR thermocycler which is configured to amplify and detect nucleic acids associated with a panel of microorganisms in the sample,
(iii) uploading the analysis data from the thermocycler to a machine learning platform, and
(iv) providing, at the retail point of sale, a profile of the consumer's skin microbiome and a personalized skincare routine for the consumer, generated from the acquired analysis data using the machine learning platform.

### DETAILED DESCRIPTION OF THE INVENTION

As used herein, “skin care” means regulating and/or improving cosmetic qualities of the skin. These qualities are subject to regulation and/or improvement both in healthy subjects as well as those which present diseases or disorders of the skin (such as atopic dermatitis, acne vulgaris, psoriasis or lichen planus).

Examples of skincare benefits in the context of this invention include providing a smoother, more even texture; improving the elasticity or resiliency of the skin; improving the firmness of the skin; reducing the oily, shiny, and/or dull appearance of skin; improving the hydration status or moisturization of the skin, improving the appearance of fine lines and/or wrinkles; improving skin exfoliation or desquamation; plumping the skin; improving skin barrier properties; improving skin tone; reducing the appearance of redness or skin blotches and improving the brightness, radiancy, or translucency of skin.

In step (i) of the method of the invention, a sample of the skin microbiome is collected from a skin surface of the consumer in a non-invasive manner. Typically, this involves the use of a sterile cotton tip swab, which is rolled over the surface of the skin using moderate pressure and circular motions.

The skin surface of the consumer is preferably a facial skin surface such as one or more of forehead, periorbital, cheek, perioral, chin, and nose skin surfaces.

In step (ii) of the method of the invention, the DNA from the sample is extracted and the extracted DNA is analyzed using a portable qPCR thermocycler.

During DNA extraction, efficient lysis of the microbial cell wall is important to obtain an optimum yield of DNA from the sample of the skin microbiome. Cell lysis methods include physical (heat), mechanical (sonication, bead-beating), chemical (pH, detergents), and enzymatic disruption of the microbial cell wall, and different methods are often combined. Mechanical disruption methods such as bead-beating can enhance nucleic acid yield by effectively lysing not only Gram-negative but also Gram-positive bacteria, which have a thick cell wall. The sample is placed in a tube with grinding beads and subjected to high energy mixing. The sample is then typically centrifuged, and the lysate recovered from above the beads. A filtration-based method may also be used, such as the Biomeme M1 Sample Prep Cartridge™ (Biomeme Inc., Philadelphia, USA), where nucleic acid binds to a silica membrane inside a piercing tool attached to a syringe. The sample is pumped through the membrane along the sealed cartridge chambers which contain lysis buffer, wash buffers, and an elution buffer. The advantage of the M1 extraction process is that no centrifugation is required.

Thermocyclers (also termed thermal cyclers or PCR machines) amplify DNA by regulating temperature in cyclical programs that comprise steps of DNA denaturation, primer annealing, and extension.

In qPCR (also termed quantitative PCR or real-time PCR), fluorescent labeling enables DNA amplification to be monitored in real time through monitoring of fluorescence. Fluorescence is measured after each cycle and the intensity of the fluorescent signal reflects the momentary amount of DNA amplicons in the sample at that specific time. In initial cycles the fluorescence is too low to be distinguishable from the background. However, the point at which the fluorescence intensity increases above the detectable level corresponds proportionally to the initial number of template DNA molecules in the sample. This point is called the quantification cycle and allows determination of the absolute quantity of target DNA in the sample according to a calibration curve constructed of serially diluted standard samples (usually decimal dilutions) with known concentrations or copy numbers. qPCR can also provide semi-quantitative results without standards but with controls used as a reference material. In this case, the observed results can be expressed as higher or lower multiples with reference to control.

A portable qPCR thermocycler suitable for use in the invention generally includes a housing, an amplification (or PCR) module, and a detection module. The amplification module is configured to receive an input sample and defines a reaction volume. The amplification module includes a heater such that the amplification module can perform a polymerase chain reaction (PCR) on the input sample. The detection module is configured to receive an output from the amplification module and a reagent formulated to produce a signal that indicates a presence of a target amplicon within the input sample. The amplification module and the detection module are integrated within the housing.

Preferred portable qPCR thermocyclers for use in the invention are of a size that can be held in one hand of a human operator.

Preferred portable qPCR thermocyclers for use in the invention weigh no more than 3.5 kg, more preferably no more than 3 kg, such as from 0.4 to 2.5 kg.

Portable qPCR thermocyclers suitable for use in the invention are commercially available, such as the Franklin™ (Biomeme Inc., Philadelphia, USA) which weighs less than 1 kg and can test biological samples without the need for centrifugation, the use of frozen reagents, or a mains power source. The device is capable of multiplex detection of up to three targets in each sample, where nine samples can be tested in a single run and results are delivered in less than an hour. Other commercially available portable qPCR thermocyclers which may be used in the invention include the Mic qPCR cycler (Bio Molecular Systems Pty Ltd., Upper Coomera, AU) and the Liberty 16 system (Ubiquitome Ltd, Auckland, NZ)

In step (ii) of the method of the invention, the portable qPCR thermocycler is configured to amplify and detect nucleic acids associated with a panel of microorganisms in the sample.

The panel of microorganisms is suitably made up of from 5 to 15, preferably from 8 to 10 different skin microorganisms.

For optimal skin health characterization, the panel of microorganisms includes skin microorganisms representing at least 5, more preferably at least 6 and most preferably at least 7 of the following genera/species: (i) Acinetobacter spp., (ii) Corynebacterium spp., (iii) Cutibacterium acnes, (iv) Lactobacillus spp., (v) Staphylococcus aureus, (vi) Staphylococcus epidermidis, (vii) Staphylococcus hominis, (viii) Staphylococcus capitis and (ix) Streptococcus spp.

Ideally, the panel of microorganisms is made up of 9 different skin microorganisms, 5 representing each of the following genera/species: (i) Acinetobacter spp., (ii) Corynebacterium spp., (iii) Cutibacterium acnes, (iv) Lactobacillus iners, (v) Lactobacillus crispatus, (vi) Staphylococcus aureus, (vii) Staphylococcus epidermidis, (viii) Staphylococcus hominis, and (ix) Streptococcus spp.

In step (iii) of the method of the invention, the analysis data is uploaded from the thermocycler to a machine learning platform.

Generally, electronic circuitry within the housing of the portable qPCR thermocycler (such as is described above) is adapted to transmit the analysis data to the machine learning platform through wired or wireless communication interfaces such as USB or Bluetooth®.

The machine learning platform utilizes one or more algorithms to generate a profile of the consumer's skin microbiome and a personalized skincare routine for the consumer based on the transmitted analysis data. The algorithms may be stored and implemented on board a computing device located at the retail point of sale (such as a smartphone, tablet, laptop, desktop, or workstation) or the algorithms may be provided from a remotely located server to which the analysis data is transmitted via a cloud service or web service. The algorithms can be based on a trained machine learning model such as an anomaly detection model or a convolutional neural network.

Additional analysis data on consumer skin attributes may also be used to enhance personalization of the routine. The machine learning platform may for example acquire skin imaging data captured by a camera-enabled mobile device (such as the consumer's own smartphone or tablet) and utilise one or more computer vision algorithms to analyse attributes such as dryness, ageing, acne, oiliness and enlarged pores, melasma and dark spots and dullness. The attributes may be scored and then normalized to an index after comparing with a reference data set of other users.

In step (iv) of the method of the invention, a profile of the consumer's skin microbiome and a personalized skincare routine for the consumer, generated from the acquired analysis data using the machine learning platform, is provided at the retail point of sale.

Preferably step (iv) is executed within 90 minutes and more preferably within 60 minutes of receipt of the sample of the skin microbiome in step (i).

The profile may indicate imbalances in the consumer's skin microbiome, for example if certain members of the panel of microorganisms are identified in the sample at levels which deviate from the normal reference levels established for these microorganisms in healthy skin microbiomes. The profile may also include a general categorization of the consumer's skin microbiome based on the relative abundances of certain members of the panel of microorganisms which are identified in the sample, for example “Cutibacterium acnes-rich,” “Lactobacillus-rich,” or “Staphylococcus-rich.” The profile may also include an indicator of risk of skin conditions that are associated with a signature microbial profile. Advantageously this may be before the phenotypic outbreak of such skin conditions, thereby enabling preventative interventions.

A personalized skincare routine may, for example, recommend the topical administration of a cosmetic composition which is designed to shift the consumer's skin microbiome profile towards a healthy equilibrium, depending on the consumer's skin microbiome profile.

Accordingly, a topical cosmetic composition for use in the invention may include one or more microbiome balancing active ingredients in a cosmetically acceptable vehicle.

Suitable microbiome balancing active ingredients for use in the invention include prebiotics which can selectively enhance the growth and/or activity of a target skin microorganism such as Staphylococcus epidermidis. Examples of prebiotics for S. epidermidis include pimelic acid (and/or its anhydride), sucrose, lactose, ribose, maltose, mannose, saccharide isomerate and glycerol and mixtures thereof.

Alternatively, a microbiome balancing ingredient may selectively reduce the growth and/or activity of a target skin microorganism such as Cutibacterium acnes in acne-affected skin. An example of such an ingredient is a blend of thymol and terpineol.

Mixtures of any of the above-described materials may also be used.

A topical cosmetic composition for use in the invention may additionally include one or more skincare actives which are designed to target specific skin attributes of the consumer, based on the image analysis described above. Examples of such skincare actives include vitamins, minerals and/or antioxidants, emollients, skin brightening agents, sunscreens, anti-irritants, exfoliating agents and mixtures thereof.

The term “cosmetically acceptable” means that the vehicle is suitable for topical application to the skin, has good aesthetic properties, is compatible with any other ingredients, and will not cause any safety or toxicity concerns.

The vehicle may comprise an aqueous phase, an oil phase, an alcohol, a silicone phase, or a mixture thereof, and may be in the form of an emulsion. Emulsions can have a range of consistencies including thin lotions (which may also be suitable for spray or aerosol delivery), creamy lotions, light creams, and heavy creams.

Topical cosmetic compositions for use in the invention may also be formulated in a single-phase carrier such as water and/or one or more water miscible organic liquids.

Topical cosmetic compositions for use in the invention may also be formulated in solid forms such as gels or sticks.

Combinations of any of the above-described product forms may also be used.

1. A method for providing skin analysis, and personalized skincare information based on that analysis, to a consumer at a retail point of sale, the method comprising:
(i) at the retail point of sale, receiving a sample of skin microbiome, which is collected from a skin surface of the consumer in a non-invasive manner,
(ii) extracting DNA from the sample of skin microbiome and analysing the extracted DNA using a portable qPCR thermocycler, wherein the portable qPCR thermocycler is configured to amplify and detect nucleic acids associated with a panel of microorganisms in the sample,
(iii) uploading the analysis data from the thermocycler to a machine learning platform, and
(iv) providing, at the retail point of sale, a profile of the consumer's skin microbiome and a personalized skincare routine for the consumer, generated from the analysis data using the machine learning platform, wherein the portable qPCR themocycler is capable of multiplex detection of up to three targets in each sample, where nine samples can be tested in a single run, and in which the panel of microorganisms is made up of 9 different skin microorganisms, representing each of the following genera/species: (i) Acinetobacter spp., (ii) Corynebacterium spp., (iii) Cutibacterium acnes, (iv) Lactobacillus iners, (v) Lactobacillus crispatus, (vi) Staphylococcus aureus, (vii) Staphylococcus epidermidis, (viii) Staphylococcus hominis, and (ix) Streptococcus spp.

2. The method according to claim 1, in which wherein the portable qPCR thermocycler weighs from 0.4 to 2.5 kg.

3. The method according claim 1, wherein step (iv) is executed within 90 minutes of receipt of the sample of skin microbiome in step (i).
